SEQUENCE LISTING (82 sequences)

SEQ ID NO: 1 - protein, 6 aa, synthetic construct
  REGION 1..6: note = Synthetic
QDVNTA

SEQ ID NO: 2 - skipped

SEQ ID NO: 3 - protein, 9 aa, synthetic construct
  REGION 1..9: note = Synthetic
QQHYTTPPT

SEQ ID NO: 4 - protein, 8 aa, synthetic construct
  REGION 1..8: note = Synthetic
GFNIKDTY

SEQ ID NO: 5 - protein, 8 aa, synthetic construct
  REGION 1..8: note = Synthetic
IYPTNGYT

SEQ ID NO: 6 - protein, 13 aa, synthetic construct
  REGION 1..13: note = Synthetic
SRWGGDGFYAMDY

SEQ ID NO: 7 - protein, 107 aa, synthetic construct [sequence omitted]

SEQ ID NO: 8 - protein, 120 aa, synthetic construct [sequence omitted]

SEQ ID NO: 9 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 14 aa, synthetic construct
  REGION 1..14: note = Synthetic
GSSTGAVTSGNYPN

SEQ ID NO: 12 - protein, 7 aa, synthetic construct
  REGION 1..7: note = Synthetic
GTKFLAP

SEQ ID NO: 13 - protein, 9 aa, synthetic construct
  REGION 1..9: note = Synthetic
VLWYSNRWV

SEQ ID NO: 14 - protein, 5 aa, synthetic construct
  REGION 1..5: note = Synthetic
KYAMN

SEQ ID NO: 15 - protein, 19 aa, synthetic construct
  REGION 1..19: note = Synthetic
RIRSKYNNYATYYADSVKD

SEQ ID NO: 16 - protein, 14 aa, synthetic construct
  REGION 1..14: note = Synthetic
HGNFGNSYISYWAY

SEQ ID NO: 17 - protein, 109 aa, synthetic construct
  REGION 1..109: note = Synthetic
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 18 - protein, 125 aa, synthetic construct
  REGION 1..125: note = Synthetic
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 19 - protein, 249 aa, synthetic construct
  REGION 1..249: note = Synthetic
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 20 - protein, 11 aa, synthetic construct
  REGION 1..11: note = Synthetic
QASQDISNYLN

SEQ ID NO: 21 - protein, 7 aa, synthetic construct
  REGION 1..7: note = Synthetic
YTSRLHT

SEQ ID NO: 22 - protein, 9 aa, synthetic construct
  REGION 1..9: note = Synthetic
QQGNTLPWT

SEQ ID NO: 23 - protein, 5 aa, synthetic construct
  REGION 1..5: note = Synthetic
NHIIH

SEQ ID NO: 24 - protein, 17 aa, synthetic construct
  REGION 1..17: note = Synthetic
YINPYPGYHAYNEKFQG

SEQ ID NO: 25 - protein, 12 aa, synthetic construct
  REGION 1..12: note = Synthetic
DGYYRDTDVLDY

SEQ ID NO: 26 - protein, 107 aa, synthetic construct
  REGION 1..107: note = Synthetic
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 27 - protein, 121 aa, synthetic construct
  REGION 1..121: note = Synthetic
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 28 - protein, 243 aa, synthetic construct
  REGION 1..243: note = Synthetic
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 29 - protein, 504 aa, synthetic construct
  REGION 1..504: note = Synthetic
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 30 - protein, 12 aa, synthetic construct
  REGION 1..12: note = Synthetic
RASQRVNNNYLA

SEQ ID NO: 31 - protein, 7 aa, synthetic construct
  REGION 1..7: note = Synthetic
GASSRAT

SEQ ID NO: 32 - protein, 9 aa, synthetic construct
  REGION 1..9: note = Synthetic
QQYDRSPLT

SEQ ID NO: 33 - protein, 5 aa, synthetic construct
  REGION 1..5: note = Synthetic
SYYWS

SEQ ID NO: 34 - protein, 16 aa, synthetic construct
  REGION 1..16: note = Synthetic
YVYYSGTTNYNPSLKS

SEQ ID NO: 35 - protein, 10 aa, synthetic construct
  REGION 1..10: note = Synthetic
IAVTGFYFDY

SEQ ID NO: 36 - protein, 108 aa, synthetic construct
  REGION 1..108: note = Synthetic
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 37 - protein, 118 aa, synthetic construct
  REGION 1..118: note = Synthetic
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 38 - protein, 241 aa, synthetic construct
  REGION 1..241: note = Synthetic
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 39 - protein, 496 aa, synthetic construct
  REGION 1..496: note = Synthetic
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 40 - protein, 982 aa, synthetic construct
  REGION 1..982: note = Synthetic
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 17 aa, synthetic construct
  REGION 1..17: note = Synthetic
KSSQSVLDSSTNKNSLA

SEQ ID NO: 42 - protein, 7 aa, synthetic construct
  REGION 1..7: note = Synthetic
WASTRES

SEQ ID NO: 43 - protein, 9 aa, synthetic construct
  REGION 1..9: note = Synthetic
QQSAHFPIT

SEQ ID NO: 44 - protein, 5 aa, synthetic construct
  REGION 1..5: note = Synthetic
NYGMN

SEQ ID NO: 45 - protein, 17 aa, synthetic construct
  REGION 1..17: note = Synthetic
WINTYTGEPTYADKFQG

SEQ ID NO: 46 - protein, 13 aa, synthetic construct
  REGION 1..13: note = Synthetic
WSWSDGYYVYFDY

SEQ ID NO: 47 - protein, 113 aa, synthetic construct
  REGION 1..113: note = Synthetic
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 48 - protein, 113 aa, synthetic construct
  REGION 1..113: note = Synthetic
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 49 - protein, 122 aa, synthetic construct
  REGION 1..122: note = Synthetic
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 50 - protein, 122 aa, synthetic construct
  REGION 1..122: note = Synthetic
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 51 - protein, 505 aa, synthetic construct
  REGION 1..505: note = Synthetic
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 52 - protein, 530 aa, synthetic construct
  REGION 1..530: note = Synthetic
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 53 - protein, 511 aa, synthetic construct
  REGION 1..511: note = Synthetic
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 54 - protein, 14 aa, synthetic construct
GSSTGAVTTSNYAN

SEQ ID NO: 55 - protein, 7 aa, synthetic construct
GTNKRAP

SEQ ID NO: 56 - protein, 9 aa, synthetic construct
ALWYSNHWV

SEQ ID NO: 57 - protein, 5 aa, synthetic construct
TYAMN

SEQ ID NO: 58 - protein, 19 aa, synthetic construct
RIRSKYNNYATYYADSVKG

SEQ ID NO: 59 - protein, 14 aa, synthetic construct
HGNFGDSYVSWFAY

SEQ ID NO: 60 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 61 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 62 - protein, 22 aa, synthetic construct
QAVVTQEPSLTVSPGGTVTLTC

SEQ ID NO: 63 - protein, 15 aa, synthetic construct
WVQQKPGKSPRGLIG

SEQ ID NO: 64 - protein, 32 aa, synthetic construct
GVPARFSGSLLGGKAALTISGAQPEDEADYYC

SEQ ID NO: 65 - protein, 10 aa, synthetic construct
FGGGTKLTVL

SEQ ID NO: 66 - protein, 5 aa, synthetic construct
TYWIE

SEQ ID NO: 67 - protein, 17 aa, synthetic construct
EILPGSGQTDFNEKFQG

SEQ ID NO: 68 - protein, 12 aa, synthetic construct
WGYYGTRGYFNV

SEQ ID NO: 69 - protein, 10 aa, synthetic construct
RASSSVSYMH

SEQ ID NO: 70 - protein, 7 aa, synthetic construct
STSNLAS

SEQ ID NO: 71 - protein, 9 aa, synthetic construct
QQRRSFPYT

SEQ ID NO: 72 - protein, 30 aa, synthetic construct
QVQLVQSGAEVKKPGASVKVSCKASGYTFS

SEQ ID NO: 73 - protein, 14 aa, synthetic construct
WVRQAPGQRLEWMG

SEQ ID NO: 74 - protein, 32 aa, synthetic construct
RVTFTADTSSDTAYMELSSLRSEDTAVYYCTR

SEQ ID NO: 75 - protein, 10 aa, synthetic construct
WGQGTLVTVS

SEQ ID NO: 76 - protein, 23 aa, synthetic construct
EIVLTQSPATLSLSPGERATLSC

SEQ ID NO: 77 - protein, 15 aa, synthetic construct
WFQQKPGQAPRLLIY

SEQ ID NO: 78 - protein, 32 aa, synthetic construct
GIPARFSGSGSGTDYTLTISSLEPEDFAVYYC

SEQ ID NO: 79 - protein, 11 aa, synthetic construct
FGQGTKLEIKR

SEQ ID NO: 80 - protein, 107 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKR

SEQ ID NO: 81 - protein, 121 aa, synthetic construct [sequence omitted]

SEQ ID NO: 82 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

(19) United States
(12) Patent Application Publication
(10) Pub. No.: US 2025/0255818 A1
(43) Pub. Date: Aug. 14, 2025

(54) LYOPHILIZATION METHOD

(71) Applicant: AMGEN INC., Thousand Oaks, CA (US)

(72) Inventors: Philippe Lam, Belmont, MA (US); Ronak Maheshwari, Thousand Oaks, CA (US); Wisam Saad Hasan Al Bakri, Moorpark, CA (US); Sai Chakradhar Padala, Thousand Oaks, CA (US); Jahangir Ashraf, Thousand Oaks, CA (US)

(21) Appl. No.: 18/857,692

(22) PCT Filed: Apr. 25, 2023
(86) PCT No.: PCT/US23/66183

§ 371 Date: Oct. 17, 2024

Related U.S. Application Data
(60) Provisional application No. 63/334,785, filed on Apr. 26, 2022.

(51) Int. Cl. A61K 9/19; C07K 16/00

(52) U.S. Cl. CPC A61K 9/19; C07K 16/00; C07K 2317/31

(57) ABSTRACT

The disclosure provides a method of preparing a lyophilized protein composition, the method comprising (a) inducing ice nucleation in a liquid protein composition in a vial exposed to a first temperature of about −15° C. to about −5° C. for about 30 minutes to about five hours; (b) exposing the vial to a second temperature of about −25° C. to −50° C. for a second period of time; and (c) drying the composition of (b) at a third temperature of about 0° C. to about 40° C., resulting in a vial comprising a lyophilized protein composition having a fill aspect ratio of greater than or equal to about 0.75. The disclosure further provides a lyophilized protein composition prepared via the method described herein.

### CROSS REFERENCE TO RELATED APPLICATION AND INCORPORATION BY REFERENCE

This application claims the benefit of priority of U.S. Provisional Patent Application No. 63/334,785, filed Apr. 26, 2022, which is hereby incorporated by reference.

Incorporated by reference in its entirety is a computer-readable nucleotide/amino acid sequence listing submitted concurrently herewith and identified as follows: XML file named “57026_Seqlisting.xml,” 97,298 bytes created Apr. 25, 2023.

### FIELD

The disclosure relates to a method of lyophilizing a protein composition and the resulting lyophilized composition.

### BACKGROUND

Lyophilization is a manufacturing process widely used to increase the stability of pharmaceutical products by removing the water content from a liquid product via freeze-drying. Lyophilization is a batch process and is typically lengthy, contributing to more than 50% of drug product processing time. In addition, manufacturing scale lyophilizers are limited with respect to capacity; only a certain quantity of vials can be loaded in a batch, and there are limits to the amount of liquid composition that can be dispensed into the vials. Due to these constraints, lyophilized products are typically costly. See, e.g., Awotwe-Otoo et al., International Journal of Pharmaceutics, 450 (2013), 70-78; Esfandiary et al., J. Pharm. Sci., 105 (2016), 1427-1433. Efforts to increase efficiency of lyophilization processes for biopharmaceutical products are hindered by variable product attributes (e.g., inconsistent lyophilized cake structure) and potential breakage of containers during the lyophilization process, which results in expensive loss of product.

### SUMMARY

The disclosure provides a method of preparing a lyophilized protein composition, the method comprising (a) providing a vial comprising a liquid protein composition having a fill aspect ratio of greater than or equal to about 0.75; (b) inducing ice nucleation in the vial exposed to a first temperature of about −15° C. to about −5° C. for about 30 minutes to about five hours; (c) exposing the vial to a second temperature of about −25° C. to −50° C. for a second period of time; and (d) drying the composition of (c) at a third temperature of about 0° C. to about 40° C. The disclosure further provides a lyophilized protein composition prepared via the method described herein.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. As such, the terms “a” (or “an”), “one or more,” and “at least one” can be used interchangeably herein. The term “or” should be understood to encompass items in the alternative or together, unless context unambiguously requires otherwise.

It should also be understood that when describing a range of values, the disclosure contemplates individual values found within the range. For example, “a pH from about pH 4 to about pH 6,” could be, but is not limited to, pH 4.2, 4.6, 5.2, 5.5, etc., and any value in between such values. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE DRAWINGS

FIG. 1 is a line graph illustrating the surface temperature profile in 30 R vials as a function of pre-ice nucleation hold time at −7° C. shelf temperature. Thermocouples were placed at the vial's liquid surface to monitor the temperatures at the top layer of the liquid. Temperature is noted on the y-axis (° C.) while time (hr) is noted on the x-axis. The various lines correspond to vials at various locations on the lyophilization chamber shelf.

FIG. 2A is a cake resistance curve comparison for an antibody formulation in 50 cc vials (non-CIN, no ice nucleation step), 50 cc vials (CIN, including ice nucleation step), and 30 R (CIN, including ice nucleation step) configurations. Cake resistance (as measured as cm² Torr hr g⁻¹) is noted on the y-axis, while dried layer thickness (cm) is noted on the x-axis.

FIG. 2B is a bar graph comparing maximum cake resistance values for the three processing conditions described in the Example. Cake resistance (as measured as cm² Torr hr g⁻¹) is noted on the y-axis.

FIG. 3 is a scatter plot illustrating cake moisture in the 30R configuration described in the Example as a function of additional hold time of the last drying segment. Cake moisture (%) is noted on the y-axis and additional hold time (hours) when drying the product is noted on the x-axis.

FIG. 4 is a scatter plot illustrating the improvement in reconstitution time of protein product lyophilized under the conditions described herein. Reconstitution time (seconds) is noted on the y-axis, while storage time (months) at 40° C. is noted on the x-axis. The circles represent reconstitution times of lyophilized product which was lyophilized without a CIN step. The triangles represent samples lyophilized with a CIN step in 50 cc containers, while the squares represent samples lyophilized with a CIN stem in 30 R containers. The samples prepared using the methods described herein required reconstitution times of less than 60 seconds. The non-CIN samples required longer reconstitution times, sometimes more than twice the amount of time required to reconstitute the samples prepared using the method described herein.

### DETAILED DESCRIPTION

The disclosure provides a method for preparing a lyophilized protein composition. The method comprises providing a vial comprising a liquid protein composition having a fill aspect ratio of greater than or equal to about 0.75 (e.g., greater than or equal to 0.8, 0.85, 0.9, or 0.95); inducing ice nucleation in the vial exposed to a first temperature of about −15° C. to about −5° C. for about 30 minutes to about five hours; exposing the vial to a second temperature of about −25° C. to −50° C. for a second period of time; and drying the composition at a third temperature of about 0° C. to about 40° C. In various aspects, the fill aspect ratio is greater than or equal to about 1 (e.g., greater than or equal to 1.1, 1.2, 1.3, 1.4, 1.5, 1.6, 1.7, 1.8, 1.9, or 2). Remarkably, the process disclosed herein allows surprisingly high fill volume for a given vial (i.e., fill aspect ratios) while minimizing the risks of vial breakage and retaining reasonable drying times. “Fill aspect ratio” is the fill height of a vial (the height of liquid protein composition in the vial) divided by the vial internal diameter (fill aspect ratio=fill height/vial internal diameter). Previous methods required reduced volumes to be introduced into vials (a lower fill aspect ratio) to prevent vial breakage and product cake collapse during the lyophilization process. The method described herein allows higher fill aspect ratios during the lyophilization process, which provides a number of potential advantages. For example, for protein therapeutics administered in large doses, fewer vials are required for dosing. Alternatively (or in addition), the process allows use of smaller vials compared to other lyophilization methods while maintaining the same fill volume, thereby resulting in more units per batch and, thus, increased output. Use of smaller vials also provides the benefit of reducing storage space manufacturing sites and clinics. These are merely examples of advantages of the instant method, which are achieved while producing product cakes suitable for biopharmaceutical use.

The disclosure describes various conditions for use in a lyophilization process to produce a lyophilized protein composition. Generally, vials (e.g., glass vials) suitable for pharmaceutical compositions are filled with liquid protein composition and exposed to different temperatures and pressures to achieve a lyophilized product. The vial may be any size or shape suitable for use in lyophilization processes, and can be formed from a variety of materials, such as glass, metal, or plastic (e.g., polycarbonate, polystyrene, polypropylene, or polyolefine). For example, the vial may be glass or glass-like and tubular in shape. Molded glass vials are commercially available in a range of different sizes with dimensions. Indeed, various sizes of vials are commercially available (e.g., size 2 R, 4 R, 6 R, 8 R, 10 R, 15 R, 20 R, 25 R, 30 R, 50 R or 100 R). The vial may be constructed to include a suitable stopper, such as a commercially available elastomeric stopper available from, e.g., Daikyo Seiko, Ltd. or West Pharmaceutical Services, Inc. The steps described herein are conducted, in many aspects, in a lyophilization chamber or ice nucleation system. Lyophilization chambers may be run per the manufacturer's instructions suitable for pharmaceutical compositions. In various aspects of the disclosure, the lyophilization method does not comprise an annealing step. “Annealing” refers to a process in which the temperature of the formulation is cycled (e.g., from a low temperature to a higher temperature, and then back to the low temperature). Various aspects of the disclosed method allow production of a lyophilized product without such an annealing step.

The method of the disclosure comprises inducing ice nucleation in the vial (referred to herein as “ice nucleation step”). Ice nucleation may be initiated using any of a number of methods including, but not limited to, ice fog, sudden/rapid depressurization, and vacuum-induced evaporative cooling. Other methods of controlled ice nucleation include, e.g., ultrasound, gap freezing, electro freezing, temperature quench freezing, use of precooled shelf, and mechanical agitation.

In various aspects of the disclosure, ice nucleation is induced via ice fog. Ice fog involves “seeding” a supercooled solution in vials with externally-generated ice crystals. An ice fog generator is used to produce a fine ice crystal suspension which is injected into the lyophilizer chamber. The crystals from the ice fog serve as ice seeds to the supercooled liquid product in the vials. Once the ice crystals from the fog enter the partially stoppered vials and contact the surface of the supercooled liquid, ice nucleation occurs instantaneously within the vial at a specified shelf temperature. This occurs simultaneously across all vials, improving intra-batch homogeneity. Ice fog systems are available from IMA Life (Tonawanda, NY) and Millrock Technologies (Kingston, NY). Ice fog technology is further described in, e.g., Azzarella et al., BioPharm. Int., 29(12) (2017), 36-41.

In various aspects of the disclosure, ice nucleation is induced via depressurization. Rapid depressurization generally involves first pressurizing a lyophilizer chamber to 1.5 to 2 atmospheres (about 20-30 psig) using an inert gas, such as nitrogen, then rapidly (e.g., in 3 seconds or less) releasing the pressure to slightly above ambient. The rapid shift in pressure induces nucleation in the vials. Rapid depressurization systems are available from SP Scientific (Gardiner, NY) and further described in, e.g., Luoma et al., “Controlled Ice Nucleation Using ControLyo® Pressurization-Depressurization Method”, In: Ward K., Matejtschuk P. (eds) Lyophilization of Pharmaceuticals and Biologicals. Methods in Pharmacology and Toxicology. Humana Press, New York, NY, 2019, pp. 57-77.

Vacuum-induced evaporative cooling generally involves reducing lyophilization chamber pressure to just above the boiling point of the solution and allowing the enhanced evaporative cooling effect of the liquid surface to cause nucleation. Vacuum-induced evaporative cooling systems are available from HOF Sonderanlagenbau GmbH (Lohra, Germany).

Ice nucleation (e.g., via ice fog) is induced in a vial exposed to a first temperature of about −15° C. to about −5° C. (e.g., about −15° C. to about −6° C. or about −15° C. to about −7° C. or about −10° C. to about −7° C., such as about −15° C., about −14° C., about −13° C., about −12° C., about −11° C., about −10° C., about −9° C., about −8° C., about −7° C., about −6° C., or about −5° C.) for a time period of about 30 minutes to about five hours. Optionally, the ice nucleation step comprises exposing the vial to the first temperature for about 30 minutes to about two hours, e.g., about 90 minutes to about two hours. In various aspects, the vial is exposed to a first temperature of about −7° C. Optionally, this step of the method further comprises holding the ice-nucleated protein composition at the temperature for a post-nucleation period of time of up to two hours (optionally at the same temperature). For example, the post-nucleation hold time may be about 30 minutes to about 90 minutes (e.g., about 45 minutes to about 75 minutes, such as 50 minutes, 55 minutes, 60 minutes, 65 minutes, 70 minutes, or 75 minutes). In various aspects, the post-nucleation hold time is about 60 minutes.

The method further comprises exposing the vial to a second temperature of about −25° C. to about −50° C. for a second period of time (referred to herein as a “freeze step”). The second temperature may be, e.g., about −25° C. to about −45° C., about −25° C. to about −40° C., about −25° C. to about −35° C., about −35° C. to about −50° C., about −40° C. to about −50° C., or about −45° C. to about −50° C. (such as about −40° C., about −41° C., about −42° C., about −43° C., about −44° C., about −45° C., about −46° C., about −47° C., about −48° C., about −49° C., or about −50° C.). In various aspects, the freeze step comprises exposing the vial to a second temperature of about −45° C. The second period of time is optionally about one hour to about five hours, such as about two hours to about four hours. The second period of time may be about 60 minutes, about 90 minutes, about 120 minutes, about 150 minutes, about 180 minutes, about 210 minutes, or about 240 minutes (or any range comprising these endpoints). In various aspects, the second period of time is about three hours.

The rate of cooling the vials may be controlled, in various aspects of the disclosure. For example, the first temperature is optionally transitioned to the second temperature at a rate of about 0.01° C. to about 0.5° C. per minute (e.g., about 0.05° C. to about 0.45° C. per minute, about 0.1° C. to about 0.3° C. per minute, or about 0.15° C. to about 0.25° C. per minute). In various aspects, the first temperature is transitioned to the second temperature at a rate of about 0.2° C. per minute.

The method further comprises drying the composition resulting from the freeze step at a third temperature of about 0° C. to about 40° C. (referred to herein as a “drying step”), resulting in a vial comprising a lyophilized protein composition having a fill aspect ratio of greater than or equal to about 0.75. In various aspects, the third temperature utilized in the drying step is about 0° C. to about 35° C., about 0° C. to about 30° C., about 5° C. to about 40° C., about 10° C. to about 40° C., about 15° C. to about 40° C., about 20° C. to about 40° C., about 25° C. to about 40° C., about 30° C. to about 40° C., or about 35° C. to about 40° C. Optionally, the third temperature is about 0° C. to about 25° C., such as about 10° C. to about 25° C. (e.g., about 25° C.).

Optionally, the transition of the freeze step to the drying step comprises increasing the temperature at rate of about 0.2° C. to about 0.7° C. per minute and holding the vial at a temperature of about −40° C. to about-30° C. for about 15 minutes to about an hour. For example, the transition may comprise increasing the temperature at a rate of about 0.2° C., 0.3° C., 0.4° C., 0.5° C., 0.6° C., or 0.7° C. The transition may occur over a time period of, e.g., about 15 minutes, 30 minutes, 45 minutes, or 60 minutes.

The drying step may, in various aspects of the disclosure, comprise exposing the vial comprising the protein composition to an increase in temperature at rate of about 0.01° C. to about 0.5° C. per minute (e.g., about 0.05° C. to about 0.45° C. per minute, about 0.1° C. to about 0.3° C. per minute, or about 0.15° C. to about 0.25° C. per minute). In various aspects of the disclosure, the drying step comprises (1) holding the vial at a temperature of about −5° C. to about 5° C. for about 8 hours to about 12 hours and (2) holding the vial at a temperature of about 20° C. to about 30° C. for about 20 hours to about 50 hours. In exemplary aspects of the disclosure, drying step 1 comprises holding the vial at a temperature of about −5° C. to about 0° C., about 0° C. to about 5° C., or about −2° C. to about 2° C.; such as about −5° C., about −4° C., about −3° C., about −2° C., about −1° C., about 0° C., about 1° C., about 2° C., about 3° C., about 4° C., about 5° C., or any range with these endpoints. The time period for drying step 1 is, in various aspects, about 8 hours to about 10 hours, about 9 hours to about 11 hours, or about 10hours to about 12 hours; such as about 8 hours, about 9 hours, about 10 hours, about 11 hours, or about 12hours, or any range with these endpoints. In exemplary aspects of the disclosure, drying step 2 comprises holding the vial at a temperature of about 25° C. to about 30° C., about 20° C. to about 25° C., or about 23° C. to about 27° C.; such as about 20° C., about 21° C., about 22° C., about 23° C., about 24° C., about 25° C., about 26° C., about 27° C., about 28° C., about 29° C., or about 30° C., or any range with these endpoints. The time period for drying step 2 is optionally about 20 hours to about 45 hours, about 20 hours to about 40 hours, about 20 hours to about 35 hours, about 20 hours to about 30 hours, about 25 hours to about 50 hours, about 30 hours to about 50 hours, about 35 hours to about 50 hours, about 40 hours to about 50 hours, or about 45 hours to about 50 hours (e.g., about 20 hours, about 25 hours, about 30 hours, about 35 hours, about 40 hours, about 45 hours, or about 50 hours). Drying step (1) optionally comprises holding the vial at a temperature of about 0° C. for about 10 hours. Drying step (2) optionally comprises holding the vial at a temperature of about 25° C. for about 40 hours.

The method of the disclosure permits use of higher volumes of liquid composition in the vials prior to lyophilization. In this regard, the liquid protein composition may fill at least 50% of the vial volume prior to performing the ice nucleation step. The method may comprise, prior to the ice nucleation step, filling at least 50% of the volume of the vials with the liquid protein composition. The liquid protein composition may fill at least 55%, 60%, 65%, or 75% of the vial.

The method described herein optionally further comprises reconstituting the lyophilized product with an appropriate diluent (e.g., sterile water, saline, etc.). In various aspects, the method described herein produces a lyophilized product which requires a shorter reconstitution time than previous lyophilization methods (i.e., less time is required to transition the lyophilized cake into a reconstituted, liquid composition suitable for administration). In this regard, the time required to reconstitute the lyophilized product to a liquid composition is reduced by at least 25%, at least 30%, at least 40%, at least 45%, or at least 50%, compared to a lyophilized product (with the same protein) that does not include, e.g., an ice nucleation step, although this is not required.

The disclosure further provides a lyophilized protein composition prepared via the method described herein. The lyophilized product optionally requires a shorter reconstitution time (at least 25%, at least 30%, at least 40%, at least 45%, or at least 50% shorter reconstitution time) compared to a lyophilized product (with the same protein) that does not include, e.g., an ice nucleation step. Alternatively or in addition, the maximum cake resistance of the lyophilized product is reduced by at least 50% (e.g., at least 55%, at least 60%, at least 65%, at least 70%, at least 75%, at least 80%, or at least 85%) compared to a lyophilized product (with the same protein) that does not include one or more of the steps described herein (e.g., the ice nucleation step). Cake resistance is the resistance of the lyophilized product to vapor flow through the dried layer, as explained further in the Example. Cake resistance is typically characterized as cm²·Torr·h·g⁻¹. See, e.g., Jameel et al., AAPS PharmSciTech. 2021 Oct; 22(7): 221.

The lyophilized protein formulation optionally further comprises a saccharide, a surfactant, and/or a buffer. The formulation also optionally has a pH of about 3 to about 7 (or about 3.5, 4, 4.5, 5, 5.5, 6, 6.5, or 7). In some cases, the pH is about 4 to about 6. In some cases the pH of the formulation is about 4, or about 4.2. In various cases, the pH of the formulation is about 5. In some embodiments, the pH of the formulation is about 6.

In some embodiments, the protein of the lyophilized formulation is an antigen-binding protein. An “antigen-binding protein” is a protein comprising a domain that binds a specified target antigen (such as HER2 or CD3 and/or DLL3, BCMA, CD33, or STEAP1). An antigen-binding protein comprises a scaffold or framework portion that allows the antigen binding domain to adopt a conformation that promotes binding of the antigen-binding protein to the antigen.

In some embodiments, the antigen-binding protein of the lyophilized formulation is an antibody or immunoglobulin, an antigen-binding antibody fragment, or an antibody protein product comprising antigen- binding domains in a scaffold, framework, or format that allows an antigen-binding domain to adopt a conformation that promotes binding to the antigen. Antibody or immunoglobulins, antigen-binding antibody fragments, and antibody protein products are also referred to herein as “antibody constructs.” In some cases, the antigen-binding protein is an antibody. The term “antibody” refers to an intact antigen-binding immunoglobulin. An “antibody” is a type of an antigen-binding protein. The antibody can be an IgA, IgD, IgE, IgG, or IgM antibody, including any one of IgG1, IgG2, IgG3 or IgG4. In various embodiments, an intact antibody comprises two full-length heavy chains and two full-length light chains. An antibody has a variable region and a constant region. In IgG formats, a variable region is generally about 100-110 or more amino acids, comprises three complementarity determining regions (CDRs), is primarily responsible for antigen recognition, and substantially varies among other antibodies that bind to different antigens. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra). The constant region allows the antibody to recruit cells and molecules of the immune system.

The architecture of antibodies has been exploited to create a growing range of alternative formats that span a molecular-weight range of at least about 12-150 kDa and have a valency (n) range from monomeric (n=1), to dimeric (n=2), to trimeric (n=3), to tetrameric (n=4), and potentially higher; such alternative formats are referred to herein as “antibody protein products.” Antibody protein products include those based on the full antibody structure and those that mimic antibody fragments which retain full antigen-binding capacity, e.g., scFv, disulfide-bond stabilized scFv (ds-scFv), single chain antibody (SCA), single chain Fab (scFab), and minibodies (miniAbs).

An antibody construct may be “bispecific,” i.e., the antibody or antibody protein product binds two different targets (e.g., CD3 and a second, different target). A “bispecific” antibody or antibody-like product generally comprises a first binding domain and a second binding domain, wherein the first binding domain binds to one antigen or target (e.g., a target cell surface antigen), and the second binding domain binds to another antigen or target (e.g., CD3). Accordingly, the antibody construct optionally comprises specificities for two different antigens or targets. The term “target cell surface antigen” refers to an antigenic structure expressed by a cell and which is present at the cell surface such that it is accessible for an antibody construct as described herein. It may be a protein, preferably the extracellular portion of a protein, or a carbohydrate structure, preferably a carbohydrate structure of a protein, such as a glycoprotein. In various aspects, it is a tumor antigen. Multispecific antibody constructs, such as trispecific antibody constructs (including three binding domains) or constructs having more than three (e.g., four, five, or more) specificities also are contemplated.

In various aspects, the protein is a bispecific antibody construct. Bispecific antibody constructs include, but are not limited to, traditional bispecific immunoglobulins (e.g., BsIgG), IgG comprising an appended antigen-binding domain (e.g., the amino or carboxy termini of light or heavy chains are connected to additional antigen-binding domains, such as single domain antibodies or paired antibody variable domains (e.g., Fv or scFv), BsAb conjugates, and engineered constructs comprising full length antibodies. See, e.g., Spiess et al., Molecular Immunology 67(2) Part A: 97-106 (2015) and International Patent Publication No. WO 2015149077, which describes various bispecific formats and is hereby incorporated by reference. Examples of bispecific antibody constructs also include, but are not limited to, diabodies, single chain diabodies, tandem scFvs, bispecific T cell engager (BITE®) format (a fusion protein consisting of two single-chain variable fragments (scFvs) joined by a linker), BsAb fragments (e.g., bispecific single chain antibodies), bispecific fusion proteins (e.g., antigen binding domains fused to an effector moiety), and Fab2 bispecifics (collectively also termed “bispecific antibody protein products”). See, e.g., Chames & Baty, 2009, mAbs 1[6]: 1-9; and Holliger & Hudson, 2005, Nature Biotechnology 23[9]: 1126-1136; Wu et al., 2007, Nature Biotechnology 25[11]:1290-1297; Michaelson et al., 2009, mAbs 1[2]: 128-141; International Patent Publication No. WO 2009032782 and WO 2006020258; Zuo et al., 2000, Protein Engineering 13[5]:361-367; U.S. Patent Application Publication No. 20020103345; Shen et al., 2006, J Biol Chem 281[16]:10706-10714; Lu et al., 2005, J Biol Chem 280[20]:19665-19672; and Kontermann, 2012 MAbs 4(2):182, all of which are expressly incorporated herein. In addition to the above, the disclosure contemplates antibody constructs (e.g., bispecific antibody constructs) comprising half-life extending formats (HLE formats), which include, for example, a single chain Fc format (designated scFc), a hetero Fc (also designated as hetFc or heterodimeric Fc, hFc) format, and the fusion of human serum albumin (also designated as HSA or hALB).

In various aspects of the disclosure, the protein in the formulation is an antibody which binds HER2 (Uniprot Accession No. P04626-1). The antibody comprises, in various aspects, a light chain complementarity determining region 1 (LC CDR1) of SEQ ID NO: 1, LC CDR2 of SEQ ID NO: 2, LC CDR3 of SEQ ID NO: 3, heavy chain (HC) CDR1 of SEQ ID NO: 4, HC CDR2 of SEQ ID NO: 5, and HC CDR3 of SEQ ID NO: 6. The anti-HER2 antibody may comprise a light chain variable region (VL) of SEQ ID NO: 7 and/or a heavy chain variable region (VH) of SEQ ID NO: 8 or sequences at least about 90% identical thereto (e.g., 91%, 92%, 93%, 94%, 95%, 96%, 97%, 98%, 99%, or 100% identical). The anti-HER2 antibody may comprise a light chain of SEQ ID NO:9 and/or a heavy chain of SEQ ID NO: 10 or sequences at least about 90% identical thereto (e.g., 91%, 92%, 93%, 94%, 95%, 96%, 97%, 98%, 99%, or 100% identical).

Where the protein is a bispecific antibody construct, the first binding domain comprises a set of six CDRs set forth in (a) SEQ ID NOs: 20-25 (LCDR1-3 and HCDR1-3, respectively, binding BCMA (e.g., UniProt Accession No. Q02223)), (b) SEQ ID NOs: 30-35 (LCDR1-3 and HCDR1-3, respectively, binding DLL3 (e.g., UniProt Accession No. Q9NYJ7)), or (c) SEQ ID NOs: 41-46 (LCDR1-3 and HCDR1-3, respectively, binding CD33 (e.g., UniProt Accession No. P20138)). In some aspects, the first binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 26, 36, 47, or 48. In some embodiments, the first binding domain of the bispecific antibody construct comprises a VL comprising the amino acid sequence set forth in SEQ ID NO: 26, 36, 47, or 48. In some aspects, the first binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 27, 37, 49, or 50. In some embodiments, the first binding domain of the bispecific antibody construct comprises a VH comprising the amino acid sequence set forth in SEQ ID NO: 27, 37, 49, or 50. In some aspects, the first binding domain comprises (a) a VH region comprising an amino acid sequence set forth in SEQ ID NO: 27 and a VL region comprising an amino acid sequence set forth in SEQ ID NO: 26; (b) a VH region comprising an amino acid sequence set forth in SEQ ID NO: 27 and a VL region comprising an amino acid sequence set forth in SEQ ID NO: 36; or (c) a VH region comprising an amino acid sequence set forth in SEQ ID NO: 49 or 50 and a VL region comprising an amino acid sequence set forth in SEQ ID NO: 47 or 48.

In some embodiments, the second binding domain of the bispecific antibody construct comprises a set of six CDRs set forth in SEQ ID NOs: 11-16 (LCDR1-3 and HCDR1-3, respectively, binding CD3 (e.g., UniProt Accession No. P07766)). In some aspects, the second binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 17. In some aspects, the second binding domain of the bispecific antibody construct comprises a VL comprising the amino acid sequence set forth in SEQ ID NO: 17. In some aspects, the second binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 18. In some embodiments, the second binding domain of the bispecific antibody construct comprises a VH comprising the amino acid sequence set forth in SEQ ID NO: 18. In some embodiments, the second binding domain comprises a VL region comprising an amino acid sequence set forth in SEQ ID NO: 17 and a VH region comprising an amino acid sequence set forth in SEQ ID NO: 18. Anti-CD3 binding domain sequences are also provided in SEQ ID NOs: 54-59 (corresponding to LCDR1-LCDR3 and HCDR1-HCDR3, respectively) and SEQ ID NOs: 60 and 61 (light chain variable region and heavy chain variable region, respectively).

In some embodiments, the bispecific antibody construct comprises a first binding domain that binds DLL3 comprising an anti-DLL3 variable light domain comprising the amino acid sequence of SEQ ID NO: 36 and an anti-DLL3 variable heavy domain comprising the amino acid sequence of SEQ ID NO: 37, a second binding domain comprising an anti-CD3 variable light domain comprising the amino acid sequence of SEQ ID NO: 17, and an anti-CD3 variable heavy domain comprising the amino acid sequence of SEQ ID NO: 18. For example, in an embodiment, the bispecific antibody construct comprises a first binding domain comprising the amino acid sequence of SEQ ID NO: 38 and a second binding domain comprising the amino acid sequence of SEQ ID NO: 19. In some embodiments, the bispecific antibody construct comprises an amino acid sequence set forth in SEQ ID NO: 39 or 40.

In some embodiments, the bispecific antibody construct comprises a first binding domain that binds BCMA comprising an anti-BCMA variable light domain comprising the amino acid sequence of SEQ ID NO: 26 and an anti-BCMA variable heavy domain comprising the amino acid sequence of SEQ ID NO: 27, a second binding domain comprising an anti-CD3 variable light domain comprising the amino acid sequence of SEQ ID NO: 17, and an anti-CD3 variable heavy domain comprising the amino acid sequence of SEQ ID NO: 18. For example, in an embodiment, the bispecific antibody construct comprises a first binding domain comprising the amino acid sequence of SEQ ID NO: 28 and a second binding domain comprising the amino acid sequence of SEQ ID NO: 19. In some embodiments, the bispecific antibody construct comprises an amino acid sequence set forth in SEQ ID NO: 29.

In some embodiments, the bispecific antibody construct comprises a first binding domain that binds CD33 comprising an anti-CD33 variable light domain comprising the amino acid sequence of SEQ ID NO: 47 or 48 and an anti-CD33 variable heavy domain comprising the amino acid sequence of SEQ ID NO: 49 or 50, a second binding domain comprising an anti-CD3 variable light domain comprising the amino acid sequence of SEQ ID NO: 17, and an anti-CD3 variable heavy domain comprising the amino acid sequence of SEQ ID NO: 18. For example, in one embodiment, the bispecific antibody construct comprises a first binding domain comprising the amino acid sequences of SEQ ID NO: 47 and 49 or SEQ ID NOs: 48 and 50, and a second binding domain comprising the amino acid sequence of SEQ ID NO: 19. In some embodiments, the bispecific antibody construct comprises the amino acid sequence set forth in SEQ ID NO: 52 or 53.

In some aspects, the bispecific antibody construct comprises a first binding domain that binds human STEAP1. The first binding domain may comprise a set of six CDRs set forth in SEQ ID NOs: 66-71 (HCDR1-3and LCDR1-3, respectively). In some aspects, the first binding domain of the bispecific antibody construct binds STEAP1 and comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 80. In some aspects, the STEAP1-binding domain of the bispecific antibody construct comprises a VL comprising the amino acid sequence set forth in SEQ ID NO: 80. In some aspects, the STEAP1-binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 81. In some embodiments, the second binding domain of the bispecific antibody construct comprises a VH comprising the amino acid sequence set forth in SEQ ID NO: 81. In some embodiments, the STEAP1-binding domain comprises a VL region comprising an amino acid sequence set forth in SEQ ID NO: 80 and a VH region comprising an amino acid sequence set forth in SEQ ID NO: 81. In this aspect, the bispecific antibody construct may comprise an anti-CD3 binding domain comprising six CDRs comprising the sequences of SEQ ID NOs: 54-59 (corresponding to LCDR1-LCDR3 and HCDR1-HCDR3, respectively) or SEQ ID NOs: 60 and 61 (light chain variable region and heavy chain variable region, respectively). In various aspects, the bispecific antibody construct that binds STEAP1 and CD3 is a construct comprising polypeptides comprising SEQ ID NOs: 89-91 (e.g., a heavy chain comprising SEQ ID NO: 90 and a heavy chain comprising SEQ ID NO: 91 paired with common light chains of SEQ ID NO: 89). Such bispecific antibodies are further described in, e.g., International Patent Publication No. WO 2020010079, incorporated herein by reference in its entirety and in particular with respect to bispecific antibody constructs.

In some aspects of the disclosure, the protein, such an antibody or bispecific antibody construct, is present in the liquid formulation (before lyophilization) in an amount ranging from about 0.1 mg/ml to about 100 mg/ml (or about 0.1 mg/mL, 0.5 mg/mL, 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, or 100 mg/mL). For example, the protein is optionally present in the liquid formulation in an amount ranging from about 0.1 mg/mL to about 70 mg/mL. In some cases, the protein is present in the liquid formulation in an amount ranging from about 0.5 mg/ml to about 30 mg/ml (or about 0.5mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, or 30 mg/mL). In various cases, the protein is present in the liquid formulation in an amount ranging from about 1 mg/ml to about 20 mg/ml (or about 1 mg/ml, 1.5 mg/mL, 2 mg/mL, 2.5 mg/ml, 3 mg/ml, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 10.5 mg/mL, 11 mg/mL, 11.5 mg/mL, 12 mg/mL, 12.5 mg/mL, 13 mg/mL, 13.5 mg/mL, 14 mg/mL, 14.5 mg/mL, 15 mg/mL, 15.5 mg/mL, 16 mg/mL, 16.5 mg/mL, 17 mg/mL, 17.5 mg/mL, 18 mg/mL, 18.5 mg/mL, 19 mg/mL, 19.5 mg/mL. or 20 mg/mL). In some aspects, the protein is present in the liquid formulation in an amount of about 21 mg/mL.

The protein formulation of the disclosure optionally comprises a saccharide. In some embodiments, the saccharide is a monosaccharide or a disaccharide. Suitable saccharides include, for example, glucose, galactose, fructose, xylose, sucrose, lactose, maltose, trehalose, or any combination thereof. In some cases, the saccharide comprises trehalose.

In some aspects, the liquid formulation (before lyophilization) comprises saccharide at a concentration of about 1% to about 15% w/v, or about 4% to about 13% w/v, or about 6% to about 12% w/v. In some embodiments, the liquid formulation comprises saccharide at a concentration of at least 1%, at least 2%, at least 3%, at least 4%, at least 5%, at least 6%, at least 7%, at least 8%, at least 9%, at least 10%, at least 11%, at least 12%, at least 13%, or at least 14% w/v. In some embodiments, the liquid formulation comprises saccharide at a concentration of about 1%, about 2%, about 3%, about 4%, about 5%, about 6%, about 7%, about 8%, about 9%, about 10%, about 11%, about 12%, about 13%, about 14%, or about 15% w/v. In some embodiments, the liquid formulation comprises saccharide at a concentration of about 7%, about 7.5%, about 8%, about 8.5%, about 9%, about 9.5%, about 10%, about 10.5%, about 11%, about 11.5%, or about 12% w/v. In some embodiments, the liquid formulation comprises saccharide at a concentration of about 7% to about 12% w/v. In some aspects, the liquid formulation comprises saccharide at a concentration of about 9% w/v. In some embodiments, the saccharide is sucrose and is present in the liquid formulation at a concentration ranging from about 6% to about 12% w/v.

The protein formulation of the disclosure optionally comprises a surfactant. Suitable surfactants include a polysorbate, a poloxomer, a polyoxyethylene, or any combination thereof. Contemplated surfactants include polysorbate 20, polysorbate 40, polysorbate 60, polysorbate 80, poloxamer 188, poloxamer 407, triton X-100, polyoxyethylene, PEG 3350, PEG 4000, and any combination thereof. In some aspects, the surfactant comprises a polysorbate. In some cases, the surfactant is polysorbate 20.

The protein formulations described herein can comprise one surfactant or a mixture of surfactants (although this is not required). In some aspects, the liquid formulation (before lyophilization) comprises a surfactant at a concentration of about 0.001% to about 5% w/v (or about 0.001% to about 0.5%, or about 0.004 to about 0.5% w/v or about 0.001 to about 0.01% w/v or about 0.004 to about 0.01% w/v). In some aspects, the liquid formulation comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some aspects, the liquid formulation comprises a surfactant at a concentration of about 0.001% to about 0.5% w/v. In some aspects, the liquid formulation comprises a surfactant at a concentration of about 0.001 to about 0.01% w/v. In some aspects, the liquid formulation comprises a surfactant at a concentration of about 0.001 to about 0.01% w/v. In some aspects, the liquid formulation comprises a surfactant at a concentration of about 0.001%, about 0.002%, about 0.003%, about 0.004%, about 0.005%, about 0.006%, about 0.007%, about 0.008%, about 0.009%, about 0.01%, about 0.05%, about 0.1%, about 0.2%, about 0.3%, about 0.4%, to about 0.5% w/v. In some aspects, the liquid formulation comprises a surfactant at a concentration of about 0.001% to about 0.01% w/v.

The protein formulation of the disclosure optionally comprises a buffer. Suitable buffers include acetate buffers, glutamate buffers, citrate buffers, lactate buffers, succinate buffers, tartrate buffers, fumarate buffers, maleate buffers, histidine buffers, phosphate buffers, 2-(N-morpholino) ethanesulfonate buffers, or any combination thereof. In some cases, the buffer comprises histidine.

Buffering agents are often employed to control pH in the formulation. In some aspects, the buffer is added in a concentration that maintains pH of the liquid formulation of about 3 to about 7, or about 4 to about 6, about 4 to 5, or about 5 to about 6, or about 6 to about 6.5. The effect of pH on formulations may be characterized using any one or more of several approaches such as accelerated stability studies and calorimetric screening studies (Remmele R. L. Jr., et al., Biochemistry, 38(16): 5241-7 (1999)).

The buffer system (when present in the protein formulation) is selected to be physiologically compatible and to maintain a desired pH. The buffer may be present in the liquid formulation (before lyophilization) at a concentration between about 0.1 mM and about 1000 mM (1 M), or between about 5 mM and about 200 mM, or between about 5 mM to about 100 mM, or between about 10 mM and 50 about mM. Suitable buffer concentrations encompass concentrations of about 200 mM or less. In some aspects, the buffer in the liquid protein formulation (before lyophilization) is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM or about 5 mM. In some aspects, the concentration of the buffer is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some aspects, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some aspects, the concentration of the buffer is between 5, 6, 7, 8, 9, 10, 11, 12, 13, 14, 15, 16, 17, 18, 19, 20, 30, or 40 mM and 50 mM.

In various aspects, the lyophilized product comprises about 420 mg of protein comprising SEQ ID NO: 9 and 10, about 381.8 a,a-trehalose dihydrate, about 9.5 mg L-histidine HCl monohydrate, about 6.1 mg L-histidine, and about 1.7 mg polysorbate 20. Reconstitution with 20 ml of the appropriate diluent (BWFI or SWFI) yields a solution containing 21 mg/ml of the protein that delivers 20 mL (420 mg of the protein), at a pH of approximately 6.

As an additional aspect, kits are provided which comprise a lyophilized protein composition described herein packaged in a manner which facilitates administration to subjects. In one aspect, the kit includes a lyophilized protein composition described herein packaged in a container such as a sealed bottle, vessel, single-use or multi-use vial, prefilled device (e.g. syringe), or prefilled injection device, optionally with a label affixed to the container or included in the package that describes use of the lyophilized protein composition. In one aspect, the pharmaceutical composition is packaged in a unit dosage form. The kit may include a device suitable for administering the reconstituted protein composition according to a specific route of administration, although this is not required. For example, the disclosure provides a dual chamber device for delivering a reconstituted protein composition disclosed herein to a subject in need thereof. Dual chamber devices are combination products containing the lyophilized protein composition disclosed herein and a diluent in two separate chambers of the device. Prefilled dual chamber devices are combination products containing freeze-dried drug and diluent in two separate chambers of the device. Suitable dual chamber devices for use with the instant disclosure are described in the art. See for example, Ingle R., Fang W. (2021). Int. Journal of Pharmaceutics 597, 12031.

The formulation described herein is useful as a pharmaceutical formulation in the treatment or amelioration of cancer in a subject in need thereof. The terms “subject in need” or those “in need of treatment” include subjects already afflicted with the disorder, as well as those in which the disorder is to be prevented. The “subject in need” or “patient” includes human and other mammalian subjects that receive either prophylactic or therapeutic treatment. “Treatment” does not require complete remission or eradication of the disease; any improvement in the disease and/or improvement in the symptoms associated with the disease are contemplated. For example, a therapeutic response would refer to one or more of the following improvements in the disease: (1) a reduction in the number of neoplastic cells; (2) an increase in neoplastic cell death; (3) inhibition of neoplastic cell survival; (4) inhibition (i.e., slowing to some extent, preferably halting) of tumor growth or appearance of new lesions; (5) slowing of disease progression; (6) an increased patient survival rate; (7) downgrade of stage of a cancer (e.g., Stage 2 to Stage 1); and/or (8) some relief from one or more symptoms associated with the disease or condition. The composition may also be administered to achieve disease prevention or slowing of onset, e.g., the avoidance of an occurrence or re-occurrence a tumor or cancer. Disease state is monitored by, e.g., clinical examination, X-ray, computerized tomography (CT, such as spiral CT), magnetic resonance imaging (MRI), positron emission tomography (PET), ultrasound, endoscopy and laparoscopy, tumor marker levels (e.g., carcinoembryonic antigen (CEA)), cytology, histology, tumor biopsy sampling, and/or counting of tumor cells in circulation. These methods also are typically used to diagnose and stage cancer.

The disclosure provides a method of treating cancer, comprising administering to a subject in need thereof a therapeutically effective amount of a reconstituted composition based on the lyophilized formulation described herein. In certain embodiments, the subject is a human. In certain aspects, the cancer is a solid tumor. In some embodiments, the cancer is brain cancer, bladder cancer, breast cancer (e.g., triple negative breast cancer), clear cell kidney cancer, cervical cancer, colon and rectal cancer, endometrial cancer, gastric cancer, head/neck squamous cell carcinoma, lip and oral cancer, liver cancer, lung squamous cell carcinoma, melanoma, mesothelioma, non-small-cell lung cancer (NSCLC), non-melanoma skin cancer, ovarian cancer, oral cancer, pancreatic cancer, prostate cancer, neuroendocrine prostate cancer, renal cell carcinoma, sarcoma, small-cell lung cancer (SCLC), Squamous Cell Carcinoma of the Head and Neck (SCCHN), or thyroid cancer. In some aspects, the cancer is acute lymphoblastic leukemia (ALL), acute myeloid leukemia (AML), chronic lymphocytic leukemia (CLL), or chronic myeloid leukemia (CML). In some aspects, the cancer is diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), or small lymphocytic lymphoma (SLL). The disclosure also provides use of a reconstituted composition based on the lyophilized formulation in a method of treating cancer, as well as use of the lyophilized formulation in the preparation of a medicament for treating cancer.

Preferably, the pharmaceutical formulation is administered parenterally, e.g., intravenously, subcutaneously, intratumorally, or intramuscularly. Parenteral administration may be achieved by injection, such as bolus injection, or by infusion, such as continuous infusion. Administration may be achieved via depot for long-term release. In some embodiments, the formulation is administered intravenously by an initial bolus followed by a continuous infusion to maintain therapeutic circulating levels of drug product. In some embodiments, the formulation is administered as a one-time dose. Pharmaceutical formulations may be administered using a medical device. Examples of medical devices for administering pharmaceutical formulations are described in U.S. Pat. Nos. 4,475, 196; 4,439, 196; 4,447,224; 4,447, 233; 4,486,194; 4,487,603; 4,596,556; 4,790,824; 4,941,880; 5,064,413; 5,312,335; 5,312,335; 5,383,851; and 5,399,163.

The Example below illustrates representative features of the disclosure. From the description of these aspects, other aspects of the invention can be made and/or practiced based on the description provided below. The methods involve use of molecular biological techniques described in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. The example serves only to illustrate the invention and is not intended to limit the scope of the invention in any way.

### EXAMPLE

This Example describes a process for preparing a lyophilized protein composition using the materials and methods described above.

Previously frozen drug substance was thawed at room temperature for 24 hours. The drug substance comprised an antibody comprising heavy and light chains of SEQ ID NOs: 10 and 9, respectively. A target fill volume of 20 mL was deposited in either 50 cc or 30 R glass vials, and fill weights were verified at the beginning of fills. Filled vials were placed in trays, manually partially stoppered, and the trays were loaded onto lyophilizer shelves. Procedures appropriate for the specific lyophilizer used were followed for experiments involving conventional recipes (i.e., loading, equilibration, freezing, primary, and secondary drying). For lyophilization processes involving induction of ice nucleation, a Millrock FreezeBooster-equipped lyophilizer was used. The FreezeBooster is an external unit that is connected to the main lyophilizer chamber and converts water into ice-fog which was then injected inside the lyophilizer chamber and eventually inside the vials, which were partially stoppered. The ice nucleation cycles included a pre-freezing temperature equilibration step to bring the product temperature below its normal freezing point prior to the ice nucleation phase. Subsequent to nucleation, various freezing ramp rates were used. Drying phases were conducted in the same manner for runs which included an ice nucleation step and runs which did not include an ice nucleation step. At the end of each lyophilization run, the chamber pressure was increased to 500 Torr by backfilling with clean, dry nitrogen, and the vials were completely stoppered via hydraulic ram. The shelf temperature was set to 5° C. Post-unloading, the vials were manually sealed using a pneumatic crimper and stored at 2-8° C. 30 R vials were used during initial runs, and the parameters were applied to 20 ml of drug substance in 50 cc vial. Stability profiles were generated using both vial configurations.

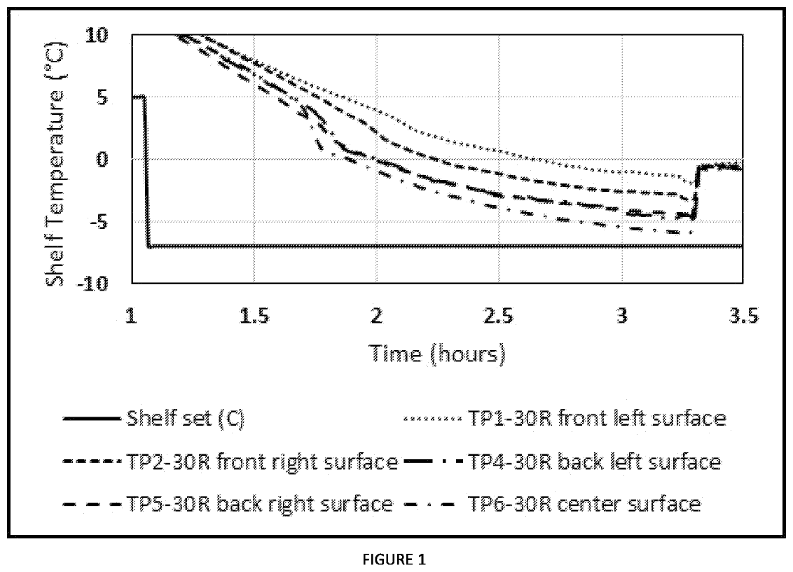

Hold time prior to ice nucleation—When ice-fog crystals are introduced inside the vial, ice-nucleation occurs at the top layer and propagates throughout the vial. A hold time prior to the nucleation step allows the product to equilibrate at a specific temperature before the introduction of ice-fog, which helps prevent ice-crystals from melting after nucleation. If the liquid surface temperature is not low enough (supercooled), the ice crystals can melt before ice can propagate throughout the vial. Hold times ranging from 0-120 minutes were tested for the 30 R configuration. Thermocouples were placed at the liquid surface to monitor the temperature at the top layer as a function of hold times as shown in FIG. 1. For a shelf temperature of −7° C., it was observed that liquid surface temperatures of 0° C. benefited from more than 60 min of hold time. Also, after considering variability in the shelf and product temperatures, a two hour hold time was adopted to ensure that the product temperature at the surface was below 0° C. prior to injecting ice-fog inside the vials. The generation of ice-fog and injection of ice-fog inside the chamber by the FreezeBooster unit takes >10 minutes. Therefore, 110 min of hold time, excluding time for the ice nucleation step itself, was selected for the pre-ice nucleation hold time for further experiments.

Ice-nucleation temperature—Higher ice nucleation temperatures achieve advantageous crystal structures and sizes and reduce cake resistance. However, when ice nucleation temperature is too high, ice fog crystals can melt. Temperatures ranging from −10° C. to −5° C. were evaluated using the 30 R configuration and batch sizes of >200 vials. The process was evaluated via thermocouple profiles and the cake appearance. Samples that were successfully nucleated under the ice nucleation conditions showed a very distinctive dried cake appearance exhibiting shiny crystalline features, and were easily distinguished from vials that did not undergo an ice nucleation step.

With pre-ice nucleation hold times of >60 min (excluding the ice nucleation step), all the vials were successfully nucleated at temperatures of −10° C. and-7° C. However, when a temperature of −5° C. was used, one of the thermocouple vials failed to nucleate for the particular drug substance used, based on the thermocouple profile. Also, based on the visual inspection, 18 out of 215 vials showed non-ice nucleated type cake appearance. Thus, while −5° C. achieved nucleation in many of the vials, −7° C. was selected for further study, as it was the highest temperature at which 100% nucleation was observed.

Post-ice nucleation hold time—While not required, additional hold time after ice nucleation, can further reduce cake resistance due to the generation of larger ice crystals. However, post ice-nucleation hold time is preferably minimized given its potential to impact product quality attributes. Post-nucleation hold times of 0 and 60 minutes were tested to assess whether there was any difference in product temperature during sublimation and primary drying profiles. A higher product temperature range (−22.5° C. to −20.5° C.) was observed during sublimation when the product had not been held for any additional time at the ice nucleation temperature as compared to the product temperature range (−23° C. to −21.5° C.) when a 60 min post-nucleation hold step had been used. No additional impact on the product quality and stability was observed. The difference in the product temperature ranges suggest that the post-nucleation hold time may be advantageous in at least some situations, allowing the formation of large ice crystal structures and reducing cake resistance. As such, 60 min of post-nucleation hold time was selected for future studies.

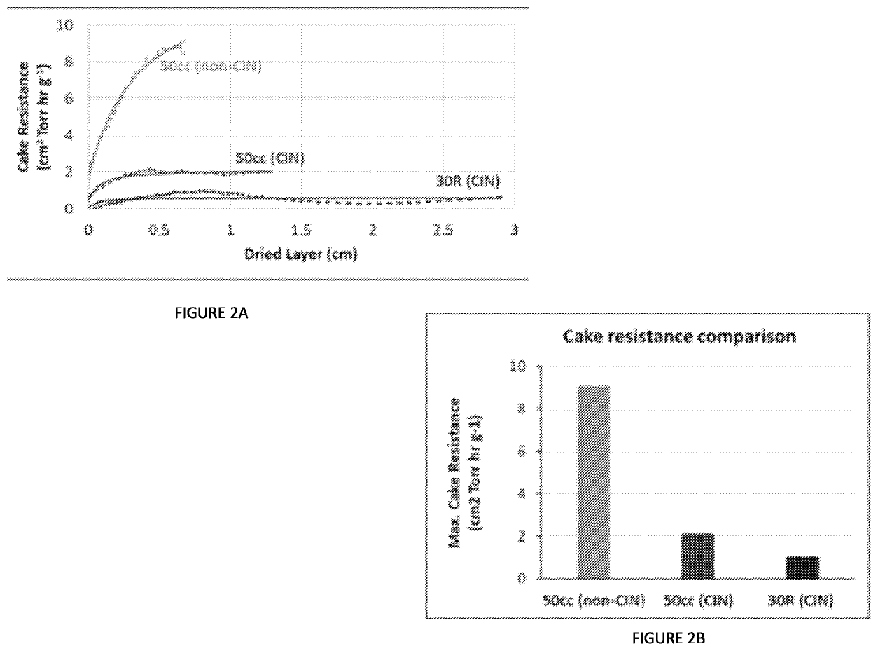

The ice nucleation conditions described increased ice crystal structure size and, thus, lower cake resistance to vapor flow as compared to processes which did not include a nucleation step. As shown in FIGS. 2A and 2B, a significant decrease in the cake resistance was observed when an ice nucleation step was performed as compared to conventional freezing processes (FIG. 2A). For comparison purposes, maximum cake resistance values were used from the three drying profiles (50 cc no nucleation step, 50 cc nucleation step, and 30 R nucleation step) and compared in FIG. 2B. More than 75% reduction in the cake resistance value was observed after addition of an ice nucleation step under the conditions described herein in the 50 cc configuration (2.2 cm² Torr.hr.g⁻¹) as compared to conventional processes (9.1 cm² Torr.hr.g⁻¹). Likewise, very low cake resistance value was achieved for the 30 R vials that underwent ice nucleation as described herein (1.0 cm² Torr.hr.g⁻¹). Difference in the cake resistance values between 50 cc and 30 R vials that underwent ice nucleation can be attributed to differences in the product temperatures during sublimation step. Maximum product temperature during sublimation in the 30 R configuration ranged from −23° C. to −21.5° C. (above collapse temperature of −26.6° C.) while the max. product temperature range for 50 cc configuration ranged from −26.4° C. to −24.7° C. Higher product temperature can lead to higher degree of micro-collapse in the cake, which can ultimately reduce the resistance to water vapor flow in the primary drying stage.

Drying step conditions also were evaluated. A chamber pressure of 150 mTorr was used to ensure sufficient heat transfer during the drying phase. Aggressive multi-segment drying steps were used for 30 R vials by changing the shelf temperatures ranging from 0° C. to 30° C. using 10 hours segments, with 5° C.-10° C. intervals. A product temperature range of −22.5° C. to −21.5° C. was observed during sublimation, which is much higher than the collapse temperature value of −26.6° C. For this product, drying above collapse temperature and higher product temperature resulted in shorter drying time with no negative impact on the cake appearance or cake moisture values compared to the samples generated using a commercial lyophilization recipe (without the nucleation step described herein). At the end of the 30° C. drying step, the cake moisture value was found to be 0.3%.

Subsequent runs were performed to assess further reduction in the total cycle time. The intermediate primary drying segments were eliminated in favor of a linear ramp from 0° C. to 25° C. at 0.03° C./min. A similar product temperature range (−23° C. to −21.5° C.) was observed with no apparent impact on cake appearance compared to the samples generated using a commercial lyophilization recipe (without the nucleation step described herein). Pirani pressure gauge data suggested that most of the primary drying was completed during the ramp from 0° C. to 25° C. and the total drying time was also reduced from 63 hours (multi-step drying) to 54hours (linear ramp drying). A slight increase in the cake moisture value (0.5%) was observed after completing drying at 25° C., as compared to multi-step (0.3%) drying ending at 30° C. A final drying segment at 25° C. was selected and used to serve the purpose of both primary and secondary drying conditions.

A short (30 min) drying step at the shelf temperature of −35° C. was added to remove any additional moisture from the chamber leftover from the ice-fog introduction, prior to initiating the sublimation of the product. As the chamber pressure of 150 mTorr was used in the lyophilization recipe, a shelf temperature of −35° C. (vapor pressure of ice is 168 mTorr) was selected to remove residual amount ice from the chamber to avoid any small increase in the temperature after completion of sublimation of external ice.

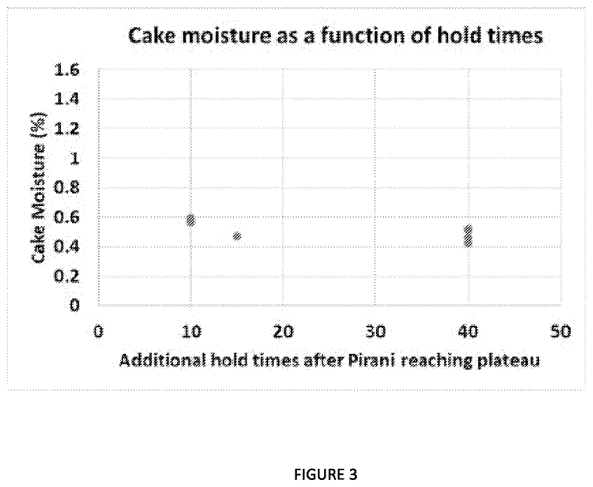

Removal of residual cake moisture during the secondary drying (SD) step depends on the SD temperature and time. After conducting secondary drying at a certain combination of temperature and time, cake moisture values reach a plateau (equilibrium) and then do not decrease substantially over time. To ensure that the cake moisture for product lyophilized using an ice nucleation step reaches an equilibrium at the end of last segment of drying, cake moisture was compared as a function of shelf temperature hold time (where time zero is when the Pirani pressure reaches a plateau). As shown in FIG. 3, cake moisture was ≤0.6% after 10 hours of hold time and only a slight decrease in the cake moisture values (0.4%-0.5%) was observed after additional hold times from 15 to 40 hours. Ten hours of additional hold time at the end of last drying segment achieved a particularly preferred cake moisture value.

After developing a process for the 30 R vial configuration, the same process was used on 50 cc vials comprising 20 ml of formulation. A product temperature ranging from −25.6° C. to −24.3° C., higher than the collapse temperature (−26.6° C.), with comparable cake appearance and cake moisture ranges (0.4%-0.6%) as product generated using commercial lyophilization process conditions was observed. The combination of an ice nucleation step at −7° C. and linear ramp (0° C. to 25° C.) drying resulted in a total drying time of 35 hours, which was significantly lower as compared to the commercial lyophilization recipe primary drying time of 100 hours at laboratory scale.

In summary, inclusion of an ice nucleation step resulted in a decrease in the total cycle times for both 50 cc and 30 R vial configurations as compared to lyophilization process without the step. In addition, manufacturing throughputs (DP vials manufactured from one batch/total lyophilization cycle time) for both 50 cc and 30 R were increased significantly as compared to processes that did not include the method steps described herein. The increase in throughput was achieved by reducing the total lyophilization cycle time and by reducing the vials footprint in case of 30 R vial, i.e., by loading a greater number of vials per batch.

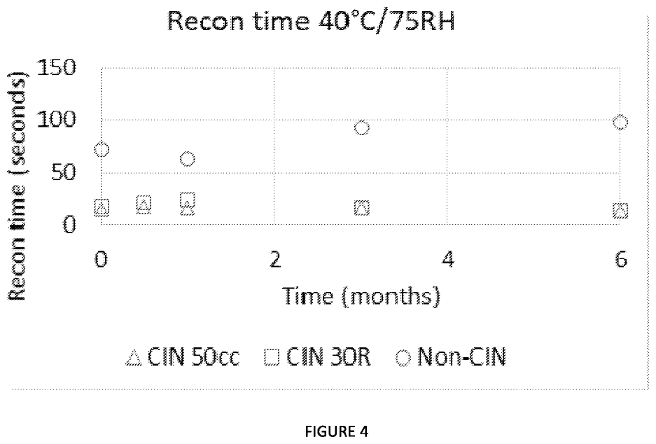

The potential impact of the ice nucleation step and drying conditions on the product quality of the drug substance was evaluated by placing both 50 cc and 30 R versions at accelerated and stressed temperature conditions (25° C./60 RH and 40° C./75 RH). The stability profiles of stability indicating assays from these two configurations were compared with stability data of drug substance lyophilized using previous conditions (i.e., without the ice nucleation step and with less aggressive drying conditions). Sub-visible particles count (HIAC), reconstitution time, cake moisture, high molecular weight (SEC), and acidic/basic peaks (CEX) attributes were studied. No negative differences in the stability trends were observed between the vials processed using the method of the disclosure and vials of the same drug substance processed using standard lyophilization procedures. Additionally, the process described herein mitigated breakage of 30 R vials comprising 20 mL fill of drug substance. Inclusion of the ice nucleation step allowed control of the ice morphology and, thus, reduced the lateral stress on the vial surface which assisted in mitigating the vial breakage compared to standard conditions. An added benefit of the method disclosed herein was a decrease in the reconstitution times of the lyophilized protein composition in both 30 R and 50 cc vials. See FIG. 4. This is attributable to, e.g., the large pore size in the cakes produced by the process of the disclosure, which facilitates the penetration of diluent during reconstitution.

To summarize, the process described herein substantially reduced total lyophilization cycle time by about 60%, allowing increased manufacturing throughput (lyophilizer vial capacity divided by hours of lyophilization time) by about 150%. Additionally, the process allowed use of a 20 mL fill volume in 30 R vials and reduced cycle time. The fill aspect ratio for the 30 R vials was 1.16 (20 mL fill volume, 20 mm fill height, 30 mm vial body diameter, 1.2 mm vial wall thickness, 27.6 vial internal diameter). Reduction in vial size and shorter cycle time resulted in an increase in the throughput by over 250% compared to previous processes. Product quality and stability of product manufactured using the method described herein in both 50 cc and 30 R vials were comparable with product manufactured using previous lyophilization recipes based on the three month stability data, at accelerated and stressed temperature conditions. Additional lyophilization runs were performed using buffer only in 2 R (2.4 mL fill volume, 13 mm fill height, 16 mm vial body diameter, 1 mm vial wall thickness, 14 vial internal diameter) and 6 R (6 mL fill volume, 17 mm fill height, 22 mm vial body diameter, 1 mm vial wall thickness, 20 vial internal diameter) vials with a fill aspect ratio of 0.93 and 0.85, respectively, and no vial breakage was observed.

All of the references cited herein, including patents, patent applications, literature publications, and the like, are hereby incorporated in their entireties by reference.

While this invention has been described with an emphasis upon preferred embodiments, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention as defined by the following claims.

What is claimed is:

1. A method of preparing a lyophilized protein composition, the method comprising
(a) providing a vial comprising a liquid protein composition having a fill aspect ratio of greater than or equal to about 0.75;
(b) inducing ice nucleation in the vial exposed to a first temperature of about −15° C. to about −5° C. for about 30 minutes to about five hours;
(c) exposing the vial to a second temperature of about −25° C. to −50° C. for a second period of time; and
(d) drying the composition of (c) at a third temperature of about 0° C. to about 40° C.

2. The method of claim 1, wherein step (b) comprises exposing the vial to the first temperature for about 30 minutes to about two hours.

3. The method of claim 2, wherein step (b) comprises exposing the vial to the first temperature for about 90 minutes to about two hours.

4. The method of any one of claims 1-3, wherein the first temperature in step (b) is about −10° C. to about −5° C.

5. The method of claim 4, wherein the first temperature in step (b) is about −7° C.

6. The method of any one of claims 1-5, wherein step (b) further comprises holding the ice-nucleated protein composition at the temperature for a post-nucleation period of time of up to two hours.

7. The method of claim 6, wherein the post-nucleation period of time is about 30 minutes to about 90 minutes.

8. The method of claim 7, wherein the post-nucleation period of time is about 60 minutes.

9. The method of any one of claims 1-8, where step (c) comprises exposing the vial to a second temperature of about −45° C.

10. The method of any one of claims 1-9, wherein the second period of time is about one hour to about five hours.

11. The method of claim 10, wherein the second period of time is about two hours to about four hours.

12. The method of claim 11, wherein the second period of time is about three hours.

13. The method of any one of claims 1-12, wherein the first temperature is transitioned to the second temperature at a rate of about 0.01° C. to about 0.5° C. per minute.

14. The method of claim 13, wherein the first temperature is transitioned to the second temperature at a rate of about 0.2° C. per minute.

15. The method of any one of claims 1-14, wherein third temperature of step (d) is about 0° C. to about 25° C.

16. The method of claim 15, wherein third temperature of step (d) is about 10° C. to about 25° C.

17. The method of claim 16, wherein third temperature of step (d) is about 25° C.

18. The method of any one of claims 1-9, wherein step (d) comprises exposing the vial comprising the protein composition to an increase in temperature at rate of about 0.01° C. to about 0.5° C. per minute.

19. The method of any one of steps 1-18, wherein step (d) comprises:
(d1) holding the vial at a temperature of about −5° C. to about 5° C. for about 8 hours to about 12 hours; and
(d2) holding the vial at a temperature of about 20° C. to about 30° C. for about 20 hours to about 50 hours.

20. The method of claim 19, wherein (d1) comprises holding the vial at a temperature of about 0° C. for about 10 hours.

21. The method of claim 19 or claim 20, wherein (d2) comprises holding the vial at a temperature of about 25° C. for about 40 hours.

22. The method of any one of claims 1-21, wherein the transition of step (c) to step (d) comprises increasing the temperature at rate of about 0.2° C. to about 0.7° C. per minute and holding the vial at a temperature of about −40° C. to about −30° C. for about 15 minutes to about an hour.

23. The method of any one of claims 1-22, wherein the liquid protein composition fills at least 50% of the vial volume prior to performing step (b).

24. The method of any one of claims 1-22, further comprising, prior to step (b), filling at least 50% of the volume of the vials with the liquid protein composition.

25. The method of any one of claims 1-24, wherein the method results in a vial comprising a lyophilized protein composition having a fill aspect ratio of greater than or equal to about 1.

26. The method of any one of claims 1-25, wherein ice nucleation is induced via ice-fog or depressurization.

27. The method of any one of claims 1-26, wherein the protein is an antibody comprising a light chain comprising the amino acid sequence set forth in SEQ ID NO: 9 and a heavy chain comprising the amino acid sequence set forth in SEQ ID NO: 10.

28. The method of any one of claims 1-26, wherein the protein is bispecific antibody construct comprising the amino acid sequence set forth in SEQ ID NOs: 29, 40, 52, or 53.

29. The method of any one of claims 1-26, wherein the protein is an antibody construct.

30. The method of claim 29, wherein the antibody construct is an antibody or a bispecific antibody construct.

31. The method of any one of claims 1-26, wherein the protein is a bispecific antibody construct comprising polypeptides comprising the amino acid sequences set forth in SEQ ID NOs: 89-91.

32. A lyophilized protein composition prepared via the method of any one of claims 1-28.

33. The lyophilized protein composition of claim 32, wherein the time required to reconstitute the lyophilized product to a liquid composition is reduced by at least 25% compared to a lyophilized product that does not include the ice nucleation step.

34. The lyophilized protein composition of claim 32, wherein the maximum cake resistance of the lyophilized product is reduced by at least 50% compared to a lyophilized product that does not include the ice nucleation step.

35. The lyophilized protein composition of any one of claims 32-34, wherein the protein is an antibody construct.

36. The lyophilized protein composition of claim 35, wherein the antibody construct is an antibody or a bispecific antibody construct.

37. The lyophilized protein composition claims 36, wherein the protein is a bispecific antibody construct comprising polypeptides comprising the amino acid sequences set forth in SEQ ID NOs: 89-91.
